(19) United States
(12) Patent Application Publication
(10) Pub. No.: US 2024/0066173 A1
(43) Pub. Date: Feb. 29, 2024

(54) DEVICE FOR DIFFUSING VOLATILE SUBSTANCES

(71) Applicant: ZOBELE HOLDING, SPA, Trento (IT)

(72) Inventors: Sergio Luque Vera, Barcelona (ES); Rubén García Fábregas, Barcelona (ES); Alberto José Sancho Marzo, Barcelona (ES)

(73) Assignees: ZOBELE HOLDING, SPA, Trento (IT); ZOBELE HOLDING, SPA, Trento (IT)

(21) Appl. No.: 18/267,999

(22) PCT Filed: Dec. 15, 2021
(86) PCT No.: PCT/EP2021/085900

§ 371 Date: Jun. 16, 2023

(30) Foreign Application Priority Data
Dec. 16, 2020 (ES) P202031256

(51) Int. Cl. A61L 9/12

(52) U.S. Cl. CPC A61L 9/12; A61L 2209/133; A61L 2209/15

(57) ABSTRACT

The diffusion device comprises a refill (1) in which liquid containing the volatile substances is housed, the refill (1) comprising: a hole (2) for the passage of the liquid; a sealing element (3) movable between a closed position in which it prevents the passage of the liquid and an open position in which it allows the liquid to pass through the hole (2); and an elastic element (4) that presses the sealing element (3) into its closed position, wherein the sealing element (3) comprises a housing (8) for housing an activation element (7) that moves the closure element (3) from the closed position to the open position against the action of the elastic element (4). It permits to provide a device that only works when the refill is original and is inserted in the correct position.

The present invention relates to a device for diffusing volatile substances.

### BACKGROUND OF THE INVENTION

Air fresheners, or in general devices for diffusing volatile substances, are very common products.

The main functions of an air freshener can be in storage, diffusion and transportation.

During storage, volatile substances are stored in an area where they are protected from external influences and are stored from production until the end of the product's shelf life.

During diffusion, the volatile substance is released into the atmosphere through dedicated diffusing means.

During transport, the volatile substance is transported from the storage area to the diffusing means.

Current diffusion devices are based on simple solutions for transport, such as wicks, or on very complex solutions, such as pumps.

There are some devices wherein the storage area is a bottle inserted upside down into the device. In these devices it is important that the connection is made correctly.

First of all, it is important that the liquid contained in the bottle does not go down just turning the bottle upside down and that it can come out only when the bottle is placed in the device.

Secondly, it is important to ensure that the liquid to be diffused is the correct one. Some volatile substances can be highly toxic. Therefore, it is important that no non-original refills can be connected to the device.

### DESCRIPTION OF THE INVENTION

Therefore, an objective of the present invention is to provide a device for diffusing volatile substances which only functions when the refill is original, and it is inserted in the correct position.

With the device for diffusing volatile substances of the invention, the above-mentioned disadvantages are solved, presenting other advantages which will be described below.

The device for diffusing volatile substances according to the present invention comprises a refill in which a liquid containing the volatile substances is housed, wherein the refill comprises:
a hole for the liquid to pass through;
a sealing element movable between a closed position in which it prevents the passage of liquid and an open position in which it allows the passage of liquid; and
an elastic element that presses the sealing element into its locked position, wherein the sealing element comprises a housing for an activation element that moves the sealing element from the closed position to the open position against the action of the elastic element.

Thanks to this feature, the liquid with the volatile substances will only come out of the refill when it is correctly positioned in the device, so that the activation element is correctly housed inside the housing.

Advantageously, the housing extends in the direction of the central axis of the sealing element, which ensures correct positioning of the activation element inside the housing.

Furthermore, the activation element preferably comprises a complementary protrusion with a recess of the sealing element, and according to a preferred embodiment, the complementary protrusion and the complementary recess are threads.

This way, when the refill is screwed in, the sealing element is displaced, and if the threads or the recess and protrusion are not complementary, the sealing element will not be displaced, and the hole will not open.

According to a preferred embodiment, the elastic element is formed by elastic membranes.

Furthermore, the hole for the passage of the liquid is preferably arranged in a partition of the refill, and the partition also comprises a cavity for the sealing element.

According to one embodiment, the sealing element is cylindrical and comprises a flange which, in the closed position, is in contact with the hole.

Furthermore, according to this embodiment, the sealing element comprises a complementary protrusion with a thread of the activation element.

According to this embodiment, the elastic element is a coil spring, and from the sealing element extend rods comprising at their distal end with respect to the sealing element abutments, which contact one end of the coil spring.

### BRIEF DESCRIPTION OF THE DRAWINGS

For a better understanding of what has been explained above, some drawings are attached in which, schematically and only as a non-limiting example, a practical case of embodiment is shown.

FIG. 1 is a perspective section view of the lower part of the refill of the device for diffusing volatile substances according to the present invention, according to a first embodiment;

FIG. 2 is a section elevation view of the refill cover of the device for diffusing volatile substances according to the present invention, according to a second embodiment, with the sealing element in its closed position;

FIG. 3 is a section elevation view of the refill cover of the device of diffusing volatile substances according to the present invention, according to a second embodiment, with the sealing element in its open position; and

FIG. 4 is a perspective section view of the lower part of the refill of the device for diffusing volatile substances according to the present invention, according to a third embodiment.

### DESCRIPTION OF PREFERRED EMBODIMENTS

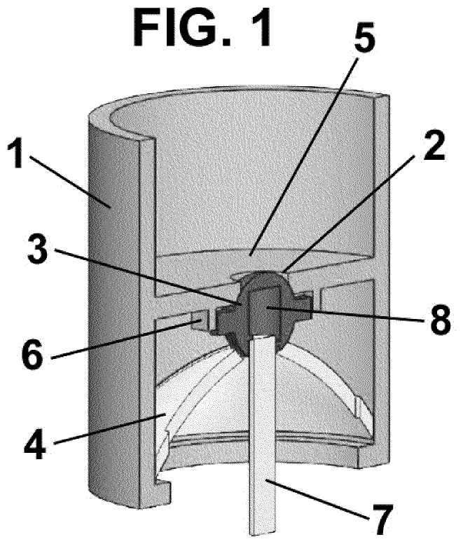

FIG. 1 shows the lower portion of a refill for a device for diffusing volatile substances, according to a first embodiment.

It should be noted that this figure shows only a portion of the refill, which contains a liquid containing the volatile substances. The rest of the refill is indicated by dashed lines, and the refill may be a bottle or any suitable container.

Said refill 1 comprises a hole 2 which can be opened or closed by means of a sealing element 3, which is movable between a closed position, in which the liquid from the refill 1 is prevented from escaping, and an open position, in which the liquid from the refill 1 is allowed to exit.

Said sealing element 3 is pushed into its sealing position by means of an elastic element 4 which, according to the embodiment shown, is formed by elastic membranes.

As can be seen from the figures, said holed 2 is arranged on a partition 5 of the refill 1, in a position centered in said partition 5, and the partition 5 also comprises a cavity 6 for the sealing element 3.

Said sealing element comprises, according to the embodiment shown, a hemispherical portion, which is the one that contacts the hole 2 in the closed position to seal it.

The device for diffusing volatile substances according to the present invention also comprises means for the diffusion of the volatile substances from the liquid contained in the refill 1, which are conventional and are not shown in the drawings.

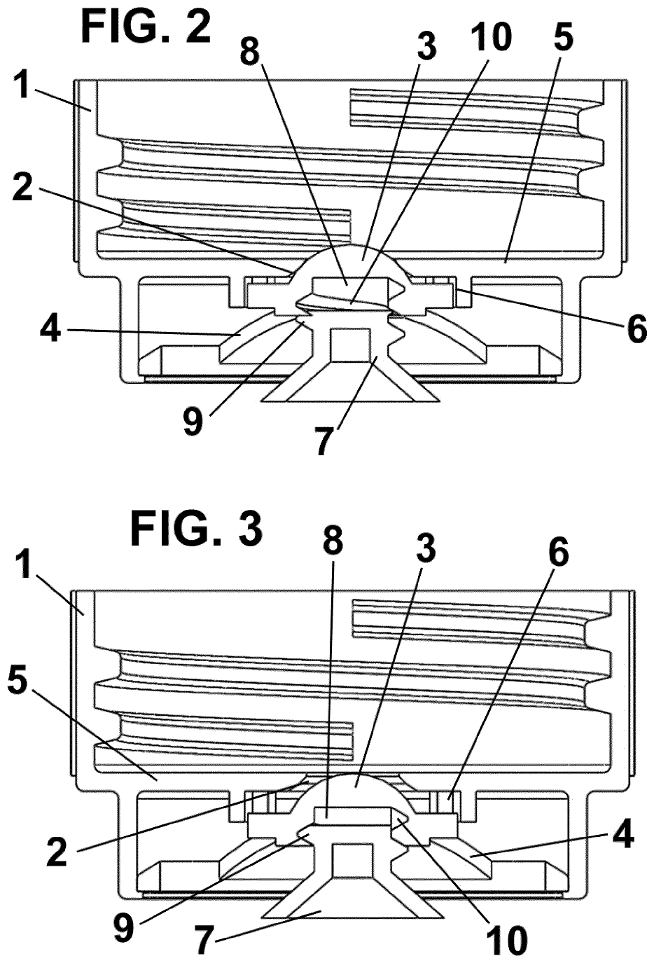

In order to change the position of the sealing element 3 from its closed position to its open position, the device for diffusing volatile substances comprises an activation element 7, which in the embodiment shown in FIG. 1 is a rod and in the embodiment shown in FIGS. 2 and 3 is a piece with a threaded end.

This activation element is placed inside a housing 8 of the sealing element 3, this housing 8 extending in the direction of the central axis of the sealing element 3.

This activation element 7 comprises a protrusion 9, such as, for example, a thread, which is complementary with a recess 10, such as a thread, of the housing 8, as shown in FIGS. 2 and 3.

Thus, in order for the sealing element 3 to be placed in its open position allowing the liquid with the volatile substances to exit through the hole 2 of the refill 1 for diffusion, the user must place the original refill 1 in its correct position. In this position, the activation element 7 of the device will be aligned with the cavity 6 of the sealing element 3 and housed therein. In addition, the protrusion 9 will be housed within the complementary, preferably threaded, recess 10 so that when the protrusion 9 is threaded into the recess 10, the activation element 7 will pull the sealing element 3 into the open position against the action of the elastic element 4.

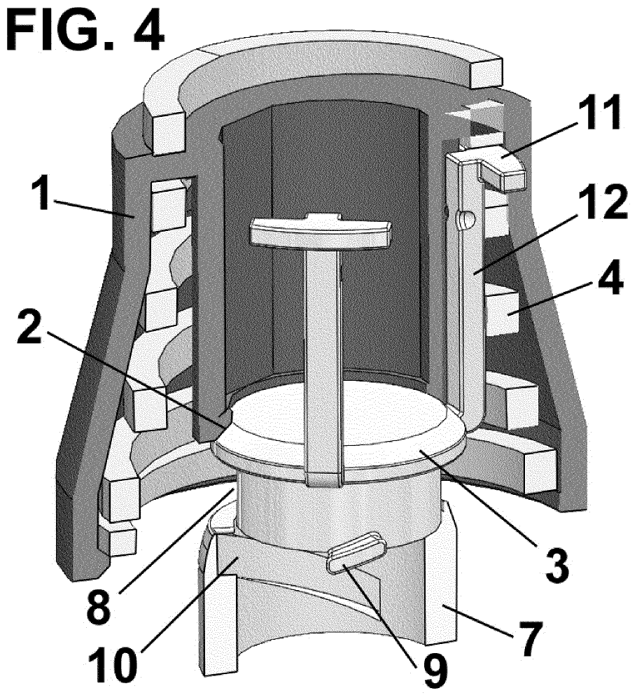

FIG. 4 shows a third embodiment of the device for diffusing volatile substances in accordance with the present invention.

For simplicity reasons, the same reference numbers are used to identify the elements that are the same or perform the same function as in the previous embodiments. In addition, only the differences from the previous embodiments are described.

According to this third embodiment, the shape of the sealing element 3 is different, in particular, it is formed from a cylinder comprising in its upper part a flange, which in its closing position is the one that closes the hole 2.

Due to this shape, the housing 8 is defined around the cylindrical wall, i.e., it is the space below said flange.

Furthermore, the protrusion 9 is placed on the cylinder wall and is housed inside the recess 10, which in this embodiment is a thread, the thread being located inside the activation element 7, which in this embodiment is a container.

A further difference with respect to the previous embodiments is the presence of rods 12 extending from the sealing element 3 and comprising an abutment 11 at the top, which is in contact with the upper part of the elastic element 4, which in this embodiment is a helical spring.

Although reference has been made to a specific embodiment of the invention, it is obvious to a person skilled in the art that the device for diffusing volatile substances described is susceptible of numerous variations and modifications, and that all the details mentioned may be replaced by other technically equivalent ones, without departing from the scope of protection defined by the appended claims.

1. A device diffusing volatile substances, comprising a refill (1) in which a liquid containing the volatile substances is housed,
characterized in that the refill (1) comprises:
a hole (2) for the passage of the liquid;
a sealing element (3) movable between a closed position in which it prevents the passage of liquid and an open position in which it allows the passage of liquid through the hole (2); and
an elastic element (4) which presses the sealing element (3) into its closed position,
wherein the sealing element (3) comprises a housing (8) for housing an actuation element (7) which moves the sealing element (3) from the closed position to the open position against the action of the elastic element (4).

2. Device for diffusing volatile substances according to claim 1, wherein the housing (8) extends in the direction of a central axis of the sealing element (3).

3. Device for diffusing volatile substances according to claim 1, wherein the activation element (7) comprises a protrusion (9) complementary with a recess (10) of the sealing element (3).

4. Device for diffusing volatile substances according to claim 3, wherein the complementary protrusion (9) and recess (10) are threads.

5. Device for diffusing volatile substances according to claim 1, wherein the elastic element (4) comprises elastic membranes.

6. Device for diffusing volatile substances according to claim 1, wherein the hole (2) for the passage of the liquid is arranged in a partition (5) of the refill (1).

7. Device for diffusing volatile substances according to claim 6, wherein the partition (5) comprises a cavity (6) for the sealing element (3).

8. Device for diffusing volatile substances according to claim 1, wherein the sealing element (3) is cylindrical and comprises a flange which, in the closed position, is in contact with the hole (2).

9. Device for diffusing volatile substances according to claim 1, wherein the sealing element (3) comprises a protrusion (9) complementary with a thread (10) of the activation element (7).

10. Device for diffusing volatile substances according to claim 1, wherein the elastic element (4) is a helical spring, and from the sealing element (3) extend rods (12) comprising at their distal end with respect to the sealing element (3) abutments (11), which contact one end of the helical spring.
